(19) United States
(12) Patent Application Publication
(10) Pub. No.: US 2020/0345881 A1
(43) Pub. Date: Nov. 5, 2020

(54) STERILIZED-LIQUEFIED GAS APPARATUS AND CONNECTIN PIPE THEREOF

(71) Applicant: ULVAC, INC., Chigasaki-shi (JP)

(72) Inventor: Tsuyoshi YOSHIMOTO, Chigasaki-shi (JP)

(21) Appl. No.: 16/759,232

(22) PCT Filed: Jul. 25, 2018
(86) PCT No.: PCT/JP2018/027919

§ 371 Date: Apr. 24, 2020

(30) Foreign Application Priority Data
Nov. 7, 2017 (JP) 2017-214520

(51) Int. Cl. A61L 9/03; F17C 5/04; A61K 33/00

(52) U.S. Cl. CPC A61L 9/03; F17C 5/04; A61K 33/00; F17C 2227/0337; F17C 2205/0352; F17C 2221/014; A61L 2101/02; F17C 2205/0323; F17C 2205/0341; A61L 2209/14; A61L 2209/111; A61L 2209/134; F17C 2250/0404

(57) ABSTRACT

A sterilized-liquefied gas apparatus of the invention includes: a liquefied gas reservoir; a source-gas supplier that supplies a source gas to the liquefied gas reservoir; a cooler that cools down an inside of the liquefied gas reservoir and thereby liquefies the source gas; a supply pipe that connects the source gas supplier and the liquefied gas reservoir; a sterilization filter provided in the supply pipe; a sterilizer that sterilizes, by a sterilizing gas, a sterilization region that is located further downstream than the sterilization filter; and a sterilizing-gas remover that removes the sterilizing gas after sterilization.

### CROSS REFERENCE TO RELATED APPLICATIONS

This application is the U.S. National Phase Application under 35 U.S.C. § 371 of International Patent Application No. PCT/JP2018/027919 filed Jul. 25, 2018, which designated the United States and was published in a language other than English, which claims the benefit of Japanese Patent Application No. 2017-214520 filed on Nov. 7, 2017, both of which are incorporated by reference herein.

### FIELD

The present invention relates to a sterilized-liquefied gas apparatus and a connecting pipe of the sterilized-liquefied gas apparatus, and particularly relates to technique preferably used when a liquefied gas such as liquid nitrogen is produced, stored therein, and supplied thereto in an aseptic condition.

### BACKGROUND

Liquefied gas such as liquid nitrogen in an aseptic condition is increasingly used in the fields of medicine, pharmaceuticals, food, and research. In accordance with this, an apparatus of producing a sterilized-liquefied gas is required.

Patent Document 1 proposes a method that sterilizes the inside of an apparatus filled with liquid nitrogen by heating by use of a high-temperature gas and maintains the sterility of the apparatus.

Patent Document 2 discloses that liquid nitrogen is sterilized by use of a filter made of a material having a resistance to extremely low temperature.

Patent Document 3 discloses that a sterilized gas is liquefied by cooling down and pipes or the like are sterilized by steam.

In contrast, an isolator having a working room in which operation for targeting a tissue-derived biomaterial is carried out is disclosed in Patent Document 4.

As disclosed in Patent Document 4, it is necessary to maintain the working room in a sterilized state in the isolator.

### PRIOR ART DOCUMENTS

### Patent Documents

(Patent Document 1) Japanese Unexamined Patent Application, First Publication No. 2000-185710

(Patent Document 2) Japanese Patent No. 4766226

(Patent Document 3) Published Japanese Translation No. 2013-531212 of PCT International Publication

(Patent Document 4) Japanese Unexamined Patent Application, First Publication No. 2009-226048

### SUMMARY

### Problems to be Solved

However, according to the technique disclosed in Patent Document 1, a large amount of energy and time is required for heating the apparatus, and the apparatus must resist both ultralow temperatures due to liquid nitrogen and high temperatures due to a high-temperature gas. Consequently, a wide range such as approximately −200° C. to +200° C. is required as heat characteristics necessary for the components forming the apparatus, and there are problems in that a range to select materials used to form the components becomes narrow, the entire configuration of the apparatus becomes complicated, and the like. Furthermore, in the technique disclosed in Patent Document 1, since a high-temperature gas is used, there is a problem in that it is difficult to connect them to the isolator as disclosed in Patent Document 4.

Additionally, in Patent Document 2, a filter that can maintain a function of, for example, sterilizing liquid nitrogen for a long period of time is not present, and practically, it is not easy to produce aseptic liquid nitrogen.

Furthermore, Patent Document 3 discloses that a sterilized gas is liquefied by cooling down and the pipes or the like are sterilized by steam. However, since a reservoir that stores liquefied gas is not provided, a supply amount of liquefied gas cannot be changed, moreover, since a high-temperature gas is used, there is a problem in that it is not possible to maintain an aseptic condition in liquid nitrogen as described hereinbelow.

In the isolator as disclosed in Patent Document 4, there is a demand for supply of aseptic liquid nitrogen. However, currently, a specific technique is not realized. The reason for this is that, as liquid nitrogen vaporizes, moisture or contaminants in air are incorporated thereinto, and therefore it is difficult to supply it to the isolator in an aseptic condition.

Particularly, in the technology related to iPS cells, there is an urgent need to realize provision of aseptic liquid nitrogen.

Furthermore, a size of each isolator as disclosed in Patent Document 4 is smaller than, for example, that of a semiconductor manufacturing facility. Therefore, facilities which supply liquid nitrogen and have a suitable size for use of an isolator are required.

Moreover, in a single plant, there is a demand that aseptic liquid nitrogen can be supplied to a plurality of isolators; however, simultaneously, there is a demand to avoid a liquid nitrogen production apparatus from being larger in size.

In such plant, since the arrangement of the isolator may be often changed, there is also a demand that aseptic liquid nitrogen can be supplied while achieving flexibility of change of such layout.

Furthermore, it is required that, the work sequence be simplified as possible, an aseptic condition be maintained, aseptic condition be strictly guaranteed, and supplying such liquid nitrogen be possible.

The invention was conceived in view of the above-described circumstances and achieves an object to provide a sterilized-liquefied gas apparatus which is used for production and supply of a sterilized-liquefied gas (liquid nitrogen), can continuously produce and continuously supply a sterilized-liquefied gas, can guarantee that liquefied gas is in an aseptic condition, the gas can be supplied by selecting a plurality of isolators or the like, and an operation process is omitted.

### Means for Solving the Problems

In order to solve the aforementioned problem, a sterilized-liquefied gas apparatus according to a first aspect of the invention, includes: a liquefied gas reservoir; a source-gas supplier that supplies a source gas to the liquefied gas reservoir; a cooler that cools down an inside of the liquefied gas reservoir to liquefy the source gas; a supply pipe that connects the source-gas supplier and the liquefied gas reservoir; a sterilization filter provided at the supply pipe; a sterilizer that sterilizes a sterilization region by a sterilizing gas, the sterilization region being located further downstream than the sterilization filter; and a sterilizing-gas remover that removes the sterilizing gas after sterilization.

In the sterilized-liquefied gas apparatus according to the first aspect of the invention, a liquefied gas that is obtained by liquefying the source gas may be liquid nitrogen.

The sterilized-liquefied gas apparatus according to the first aspect of the invention may further include a moving device that is capable of moving at least the liquefied gas reservoir.

The sterilized-liquefied gas apparatus according to the first aspect of the invention may further include: a supplier that is connected to the liquefied gas reservoir, supplies a liquefied gas stored in the liquefied gas reservoir toward a downstream side of the liquefied gas reservoir, and is capable of being hermetically-sealed; and a supply sterilizer that sterilizes the supplier.

In the sterilized-liquefied gas apparatus according to the first aspect of the invention, the supply sterilizer is capable of supplying the liquefied gas to the liquefied-gas supply object in an aseptic condition when the supplier is connected to a liquefied-gas supply object, and the supply sterilizer may be capable of carrying out aseptic treatment that sterilizes a supplier.

The sterilized-liquefied gas apparatus according to the first aspect of the invention further includes a connection sensor that detects that the supplier is connected to the liquefied-gas supply object, wherein in the case where the connection sensor determines that the supplier is connected to the liquefied-gas supply object, the supply sterilizer may be capable of starting the aseptic treatment.

The sterilized-liquefied gas apparatus according to the first aspect of the invention further includes a moving sensor that detects that the liquefied gas reservoir is in movement, wherein in the case where the moving sensor detects that the sterilized-liquefied gas apparatus is in movement, liquefying treatment may be able to be stopped.

A connecting pipe of the sterilized-liquefied gas apparatus according to a second aspect of the invention is a connecting pipe to be connected to a liquefied gas reservoir and a liquefied-gas supply object, and the connecting pipe includes: a connection portion that is connectable to the liquefied gas reservoir and the liquefied-gas supply object; and a valve capable of being hermetically-sealed, wherein in a state where an inside of the connecting pipe is hermetically sealed by the valve, the connecting pipe is connected to a vacuum pumping device that is capable of discharging gas inside the connecting pipe, and the connecting pipe is connected to a sterilizer that is capable of supply a sterilizing gas to an inside of the connecting pipe in a state where gas is discharged therefrom.

The sterilized-liquefied gas apparatus according to the first aspect of the invention includes: a liquefied gas reservoir; a source-gas supplier that supplies a source gas to the liquefied gas reservoir; a cooler that cools down an inside of the liquefied gas reservoir to liquefy the source gas; a supply pipe that connects the source-gas supplier and the liquefied gas reservoir; a sterilization filter provided at the supply pipe; a sterilizer that sterilizes a sterilization region by a sterilizing gas, the sterilization region being located further downstream than the sterilization filter; and a sterilizing-gas remover that removes the sterilizing gas after sterilization. According to this configuration, the sterilizing gas is supplied from the sterilizer to at least the sterilization filter, the supply pipe, and the inside of the liquefied gas reservoir, which is the sterilization region located further downstream than the sterilization filter. Consequently, the sterilization region is gas-sterilized, sterilizing gas, moisture, or the like which is generated due to gas sterilization is removed by the sterilizing-gas remover, the sterilizing gas is removed from the sterilization region, and sterilization treatment with respect to the sterilization region is completed. The source gas in an aseptic condition is supplied from the source-gas supplier through the sterilization filter to the liquefied gas reservoir in the aseptic condition, and the cooler cools down the inside of the liquefied gas reservoir to liquefy the source gas. Furthermore, the sterilized-liquefied gas is stored in the liquefied gas reservoir. Accordingly, it is possible to supply the sterilized-liquefied gas produced as needed to the outside.

Furthermore, in the sterilized-liquefied gas apparatus according to the first aspect of the invention, in a production step of a sterilized-liquefied gas and a sterilization step associated with the production step, there are no cases where a pressure is applied to the reservoir and the region in which the liquefied gas is produced. For this reason, it is not necessary maintain pressure resistance against a high-pressure in the region, and space-saving of the apparatus and reduction in size thereof can be achieved. Moreover, workload necessary for maintenance management is reduced, and improvement in reduction of the production cost and the supply cost can be achieved.

Particularly, it is not necessary to provide a large-scale facility such as installation of a large-scale reservoir.

Here, “aseptic” means a state where sterilization is achieved, and “sterilization” means that all of microorganisms and viruses that exist in a target object are annihilated or removed regardless of harmfulness or harmlessness. Specifically, it means that sterility assurance level (SAL) is satisfied and means SAL≤10⁻⁶ (After the sterilization operation, the probability of microorganisms to survive in the object to be sterilized is less than 1 part per million).

Moreover, the liquefied gas is a gas having a normal boiling point of −50° C. or less, and nitrogen, oxygen, liquid air and argon can be adopted.

In the first aspect of the invention, the sterilizing-gas remover carries out discharge and removal of the sterilizing gas remaining in the sterilization region and removal of generated moisture, by supplying inert gas thereto. Accordingly, after sterilization treatment is carried out due to supply of the sterilizing gas by the sterilizer, the inert gas is supplied to the sterilization region by the sterilizing-gas remover, and therefore the sterilizing gas or the like, which is adhered to at least the sterilization filter, the supply pipe, and the inside of the liquefied gas reservoir serving as the sterilization region or which is stored in the liquefied gas reservoir, is discharged to outside and removed. As a result, regeneration of contamination or bacteria due to remaining sterilizing gas, moisture, or the like is prevented, improvement in sterilization and cleanup of the sterilization region is achieved after sterilization treatment, it is possible to produce a sterilized-liquefied gas, and it is also possible to guarantee an aseptic condition of the produced liquefied gas.

Here, in the sterilizing-gas remover, as the inert gas to be supplied to the sterilization region, nitrogen gas having a high temperature higher than a room temperature, preferably, nitrogen gas of 100° C. or higher can be used.

Accordingly, as it is the same as the sterilized-liquefied gas that produces the sterilizing gas, it does not adversely affect a degree of purity of the gas to be produced even where the sterilizing gas remains, and is, therefore, preferable. Moreover, in the sterilization step, the steps of initially utilizing air, thereafter replacing air with inert gas, and carrying out sterilization is excellent in terms of cost, it is possible to avoid the above disadvantageous effect, both of the advantages can be achieved, and is, therefore, preferable. Similarly, as the types of inert gases, by using two types of gases, by switching gas supply, and by carrying out sterilization, it is also possible to obtain the same effect as that of the above.

Note that, in order to reduce an amount of time for removal treatment of sterilizing gas, it is preferable that the supply flow rate of the gas in the removal treatment of sterilizing gas be higher than the supply flow rate of the source-gas supplier when the liquefied gas is produced. Note that, by reducing a pressure of the sterilization region such as the liquefied gas reservoir, it is possible to lower the temperature of the inert gas to be supplied to the sterilization region to be lower than 100° C. For example, in the case where the pressure is reduced to 30 kpa, it is possible to set the temperature to approximately 70° C.

In the first aspect of the invention, as the sterilizer and the sterilizing-gas remover are connected to the supply pipe provided in the upstream side of the sterilization filter, the sterilizer and the sterilizing-gas remover are in a state of being connected to the upstream side of the sterilization region. Accordingly, since the entirety of the sterilization region can be subjected to the sterilization treatment and the removal treatment of sterilizing gas, the entirety of the sterilization region is in an aseptic condition, production of a sterilized-liquefied gas can be carried out, and it is also possible to guarantee an aseptic condition of the produced liquefied gas.

Additionally, in the first aspect of the invention, since the supply pipe is connected to the upper portion of the liquefied gas reservoir, the supply pipe is not necessary to penetrate through the side surface and the bottom surface of the liquefied gas reservoir. Because of this, a surface treatment on the inner surface of liquefied gas reservoir can be easily in a predetermined state.

Note that, in the sterilized-liquefied gas apparatus according to the first aspect of the invention, the surface to be in contact with a liquefied gas or the inner surface of the sterilization region is in a state that satisfies a Sanitary Standard. For this reason, it is possible to guarantee an aseptic condition of the produced liquefied gas.

Here, Sanitary Standard means a standard used for production of foods, dairy husbandry, brewage, beverage, confectionery, seafood processing, medication, cosmetics, industrial chemical product, cold beverage, beer, alcohol, meat processing, chemical agent, semiconductor, or the like.

Furthermore, since the cooler includes the freezing machine, it is easy to cool down the inside of the liquefied gas reservoir to a temperature at which a source gas is liquefied or a temperature less than the temperature, and it is possible to produce a large amount of liquefied gas which is stored inside the liquefied gas reservoir.

In the sterilized-liquefied gas apparatus according to the first aspect of the invention, as the liquefied gas that is obtained by liquefying the source gas is liquid nitrogen, it is possible to easily supply aseptic liquid nitrogen to the isolator or the like which is necessary to maintain the working room in a sterilized state and is not realized in a conventional case, while guaranteeing an aseptic condition.

Additionally, the sterilized-liquefied gas apparatus according to the first aspect of the invention includes the moving device that is capable of moving at least the liquefied gas reservoir. With this configuration, the liquefied gas (liquid nitrogen) is produced in an aseptic condition, the liquefied gas is stored in the reservoir, the reservoir is transferred after completion of production of the liquefied gas, and it is possible to optionally supply the liquefied gas to a desired device or the like while maintaining an aseptic condition. Particularly, it is possible to supply the required amount of the sterilized-liquefied gas to a plurality of supply objects such as an isolator when needed. Note that, when it is transferred, the reservoir merely functions as a so-called Dewar vessel.

Furthermore, in the sterilized-liquefied gas apparatus according to the first aspect of the invention, when it is in movement and the liquefied gas is simply supplied thereto, it is sufficient only to supply an electrical power required for a sensor and control inside the sterilized-liquefied gas apparatus, a required large electric power at all times is not necessary when the liquefied gas is produced. Accordingly, a portable configuration only by use of UPS (uninterruptible power supply) can be realized.

Moreover, the sterilized-liquefied gas apparatus according to the first aspect of the invention includes: a supplier that is connected to the liquefied gas reservoir, supplies a liquefied gas stored in the liquefied gas reservoir toward a downstream side of the liquefied gas reservoir, and is capable of being hermetically-sealed; and a supply sterilizer that sterilizes the supplier. With this configuration, the reservoir is moved to be connected to the supply object, and pipes or the like of the isolator or the like which serves as the supplier and a connection object are subjected to aseptic treatment by the supply sterilizer. Consequently, it is possible to easily supply the liquefied gas stored in the reservoir to the connection object through the aseptic supplier and the pipes or the like serving as the connection object while being in an aseptic condition. Because of this, it is possible to supply any aseptic liquefied gas to a plurality of supply objects at a desired place at an optional timing.

In addition, in the sterilized-liquefied gas apparatus according to the first aspect of the invention, the supply sterilizer is capable of supplying the liquefied gas to the liquefied-gas supply object in an aseptic condition when the supplier is connected to a liquefied-gas supply object, and the supply sterilizer is capable of carrying out aseptic treatment that sterilizes a supplier. With this configuration, only by moving the reservoir to be connected to the supply object, the aseptic treatment with respect to the supplier is started by the supply sterilizer, and it is possible to easily supply the liquefied gas stored in the reservoir to the connection object through the aseptic supplier while being in an aseptic condition. Furthermore, only by connecting the supplier to the connection object, supply of the sterilized-liquefied gas stored in the reservoir to the connection object can be automated while an aseptic condition is guaranteed. Furthermore, when any aseptic liquefied gas is supplied to a plurality of supply objects at a desired place at an optional timing, liquefied gas supply operation can be automated while guaranteeing an aseptic condition.

Additionally, the sterilized-liquefied gas apparatus according to the first aspect of the invention further includes a connection sensor that detects that the supplier is connected to the liquefied-gas supply object, wherein in the case where the connection sensor determines that the supplier is connected to the liquefied-gas supply object, the supply sterilizer is capable of starting the aseptic treatment. With this configuration, by starting aseptic treatment after connection of the connection portion to the supply object is checked by the connection sensor, the connection object serving as the supply object is prevented from being in a non-aseptic condition, and supply of the sterilized-liquefied gas stored in the reservoir can be achieved while reliably guaranteeing an aseptic condition.

Furthermore, the sterilized-liquefied gas apparatus according to the first aspect of the invention further includes a moving sensor that detects that the liquefied gas reservoir is in movement, wherein in the case where the moving sensor detects that the sterilized-liquefied gas apparatus is in movement, liquefying treatment is able to be stopped. With this configuration, since liquefying treatment is not carried out when the sterilized-liquefied gas apparatus is in movement, leakage of the liquefied gas from the reservoir can be prevented. The internal space of the reservoir or the accumulated liquefied gas can be prevented from being contaminated and becoming a non-aseptic condition due to connection of the reservoir and the external space.

A connecting pipe of the sterilized-liquefied gas apparatus according to a second aspect of the invention is a connecting pipe to be connected to a liquefied gas reservoir and a liquefied-gas supply object, and the connecting pipe includes: a connection portion that is connectable to the liquefied gas reservoir and the liquefied-gas supply object; and a valve capable of being hermetically-sealed, wherein in a state where an inside of the connecting pipe is hermetically sealed by the valve, the connecting pipe is connected to a vacuum pumping device that is capable of discharging gas inside the connecting pipe, and the connecting pipe is connected to a sterilizer that is capable of supply a sterilizing gas to an inside of the connecting pipe in a state where gas is discharged therefrom. With this configuration, when the sterilized-liquefied gas is supplied to the liquefied-gas supply object, in a state where the liquefied gas reservoir is connected to the liquefied-gas supply object, the connection portion thereof is sterilized, it is possible to supply the sterilized-liquefied gas to the liquefied-gas supply object while maintaining the accumulated sterilized-liquefied gas in an aseptic condition.

### Effects of the Invention

According to the aspect of the invention, in the sterilization region in which the liquefied gas is produced and accumulated, sterilization and removal is carried out by the sterilizing gas. For this reason, regeneration of bacteria in the sterilization region is prevented, an aseptic condition is easily maintained, and it is possible to carry out production, accumulation, and supply of the sterilized-liquefied gas. Furthermore, the effect is obtained that it is possible to provide the sterilized-liquefied gas apparatus that guarantees an aseptic condition of the produced liquefied gas and can carry out transfer and supply thereof while maintaining an aseptic condition.

### BRIEF DESCRIPTION OF THE DRAWINGS

FIG. 1 is a schematic diagram showing a sterilized-liquefied gas apparatus according to a first embodiment of the invention.

FIG. 2 is a flowchart showing steps of producing a liquefied gas in the sterilized-liquefied gas apparatus according to the first embodiment of the invention.

FIG. 3 is a schematic diagram showing a sterilized-liquefied gas apparatus according to a second embodiment of the invention.

FIG. 4 is a flowchart showing a sterilization step in a supplier of the sterilized-liquefied gas apparatus according to the second embodiment of the invention.

FIG. 5 is a schematic diagram showing a sterilized-liquefied gas apparatus according to a third embodiment of the invention.

FIG. 6 is a schematic diagram showing an example of a moving sensor of the sterilized-liquefied gas apparatus according to the third embodiment of the invention.

FIG. 7 is a schematic diagram showing a sterilized-liquefied gas apparatus according to a fourth embodiment of the invention.

### DETAILED DESCRIPTION

Hereinafter, a sterilized-liquefied gas apparatus according to a first embodiment of the invention will be described with reference to drawings.

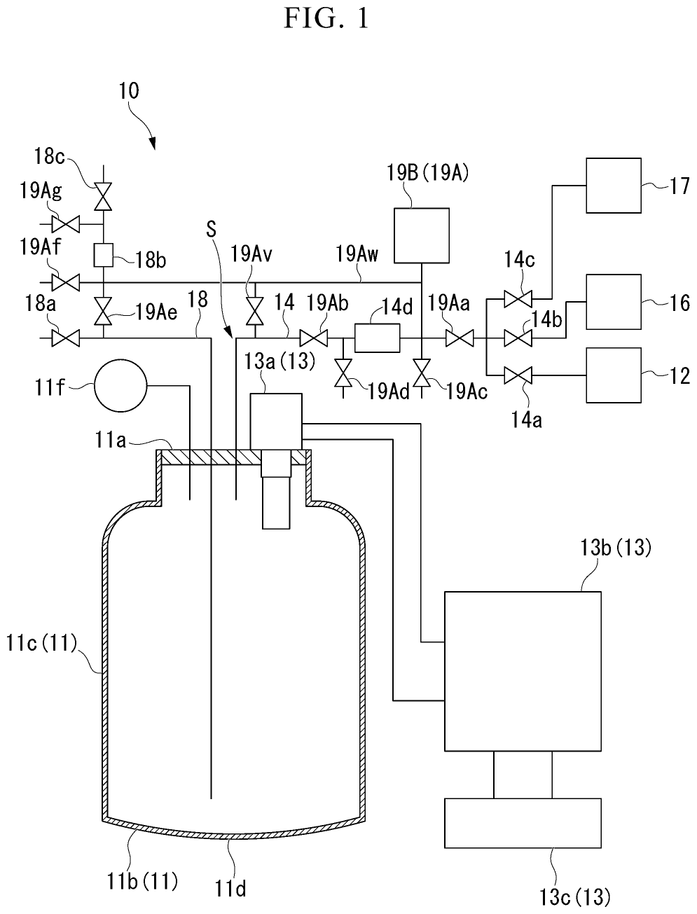

FIG. 1 is a schematic cross-sectional diagram showing a sterilized-liquefied gas apparatus according to the embodiment of the invention, reference numeral 10 in this diagram represents a sterilized-liquefied gas apparatus.

As shown in FIG. 1, the sterilized-liquefied gas apparatus 10 according to the embodiment includes a liquefied gas reservoir 11, a source-gas supplier 12, a cooler 13, a supply pipe 14, a sterilization filter 14d, a sterilizer 16, a sterilizing-gas remover 17, and a liquefied gas supplier 18 (connection pipe).

The liquefied gas reservoir 11 according to the embodiment is a hermetically-sealed reservoir as shown in FIG. 1 which is formed in a bottomed and substantially cylindrical shape, and satisfies sanitary specification which will be described later.

As shown in FIG. 1, the supply pipe 14, the connection pipe 18, and the cooler 13 are provided at the liquefied gas reservoir 11 so as to penetrate through a lid 11a serving as an upper end thereof.

A slope that smoothly lowers from the connection position between the bottom portion 11b and the side wall 11c toward the center of the bottom portion 11b is formed on a bottom portion 11b of the liquefied gas reservoir 11. A recess is formed at the central of the bottom portion 11b, and the recess forms a reservoir recess 11d. Particularly, the bottom portion 11b is formed in a curved surface shape such that a spherical surface is cut off, the strength of the liquefied gas reservoir 11 can be maintained in the case where a difference in pressure between the liquefied gas reservoir 11 and the outside occurs. As the shape of the bottom portion 11b of the liquefied gas reservoir 11, a configuration other than the configuration shown in FIG. 1 such as, for example, a flat plate shape, may be adopted.

The source-gas supplier 12 is configured to supply a source gas serving as a raw material of a liquefied gas to the liquefied gas reservoir 11 and is connected to the supply pipe 14 via a valve 14a. In the embodiment, the source-gas supplier 12 supplies nitrogen gas to the liquefied gas reservoir 11 as a source gas. The source-gas supplier 12 supplies a source gas at, for example, substantially room temperature, to the liquefied gas reservoir 11.

The source-gas supplier 12 according to the embodiment includes a nitrogen generator using an absorbing agent (PSA), effectively separate air into oxygen and nitrogen, and can supply up to 99.99% of nitrogen gas to the liquefied gas reservoir 11. In other cases, as long as a source gas can be supplied to the liquefied gas reservoir 11, the configuration of the source-gas supplier 12 is not limited to the above-mentioned configuration.

As shown in FIG. 1, the cooler 13 is a freezing machine system and is configured to include: a cooling unit 13a that functions as a mechanical freezing machine and penetrates through the lid 11a serving as an upper end of the liquefied gas reservoir 11; a compressor 13b that is connected to the cooling unit 13a and is used to supply and retrieve a refrigerant gas; and a water cooling unit 13c connected to the compressor 13b. The cooling unit 13a penetrates through the lid 11a and protrudes toward the inside of the liquefied gas reservoir 11, and is configured to liquefy the source gas due to heat exchange by the cooling unit 13a that functions as a mechanical freezing machine. The cooling unit 13a can cool down a cooling medium (for example, helium gas) supplied from the compressor 13b until being a ultralow temperature of, for example, 80K by causing the cooling medium to be subjected to Simon expansion.

The compressor 13b is configured to circulate, for example, helium gas between the cooling unit 13a and the compressor 13 and remove heat from the cooling unit 13a. The heat is discharged to by the water cooling unit 13c.

In other cases, a configuration (air cooling) may be adopted which directly discharges heat by the compressor 13b without providing the water cooling unit 13c. The cooling unit 13a that protrudes into the inside of the liquefied gas reservoir 11 is configured so that the surface thereof satisfies sanitary specification.

The downstream side of the supply pipe 14 penetrates through the lid 11a serving as an upper end of the liquefied gas reservoir 11 and is located in an axial direction at the center of the liquefied gas reservoir 11, and the supply pipe 14 is provided so as to longitudinally extend into the inside of the liquefied gas reservoir 11. A plurality of through holes are provided along the entire length of the pipe side surface of the supply pipe 14 that extends into the inside of the liquefied gas reservoir 11, and the inside of the supply pipe 14 that extends into the inside of the liquefied gas reservoir 11 is communicated with the inside of the liquefied gas reservoir 11.

The sterilization filter 14d is provided on the upstream side of the supply pipe 14. Additionally, the pipe located at the upstream side of the sterilization filter 14d is formed so as to split into three pipes. The split pipes are connected to the source-gas supplier 12, the sterilizer 16, and the dryer 17 via valves 14a, 14b, and 14c, respectively. In other cases, the valve 14a, the valve 14b, the valve 14c may be formed by three independent valves; however, as long as the structure that can switch three branches is adopted, another configuration may be adopted.

The sterilization filter 14d is provided at the supply pipe 14 and is a filter that can sterilize a source gas supplied from the source-gas supplier 12. The filter performance of the sterilization filter 14d can exhibits around a room temperature. Moreover, as described below, the sterilization filter 14d is formed by a material and in a shape which does not degrade the filter performance by a sterilizing gas supplied from the sterilizer 16. Here, the sterilization filter means a sterilization filter that can carry out sterilization.

The sterilization filter 14d, the supply pipe 14 positioned further downstream than the sterilization filter 14d, the inside of the liquefied gas reservoir 11, and the connection pipe 18 extending to a valve 18a are a sterilization region S. In order to cause a produced liquefied gas to be in an aseptic condition and supply the liquefied gas reservoir 11 the accumulated liquefied gas in an aseptic condition, the sterilization region S maintains a sterilized state from start of production of the liquefied gas to completion of supply thereof. As the sterilization filter 14d, for example, a hydrophobic PTFE membrane filter used for gas sterilization, produced by PALL Corporation, can be adopted.

The sterilization filter 14d is configured such that a plurality of fluidic channels are replaceably provided inside the sterilization filter 14d, and for example, is configured to be able to replace two or three filters in parallel.

An integrity testing device 19A that checks and inspects that the sterilization filter 14d is in a state of being capable of maintaining an aseptic condition may be provided at the sterilization filter 14d.

The integrity testing device 19A may include: a valve 19Aa provided at the upstream side of the sterilization filter 14d of the supply pipe 14; a valve 19Ab provided at the downstream side of the sterilization filter 14d of the supply pipe 14; a valve 19Ac provided to split from the supply pipe 14 at the position between the valve 19Aa and the sterilization filter 14d; a valve 19Ad provided to split from the supply pipe 14 at the position between the valve 19Ab and the sterilization filter 14d; and an integrity testing unit 19B connected to a branching position of the valve 19Ac so as to split from the supply pipe 14.

In the integrity testing device 19A, when the valves 19Aa, 19Ab, and 19Ac are in a closed state and the valve 19Ad is in an opened state, the integrity testing unit 19B supplies, for example, deionized water, and deionized water that passed through the sterilization filter 14d is collected from the valve 19Ad. It is determined whether or not the filter maintains an aseptic condition by inspecting the collected deionized water.

After the integrity testing is completed, the valves 19Ab and 19Ad are in a closed state, the valves 19Aa, 19Ac, and 19Ad are in an opened state, the valve 14c is opened, and a purge gas such as inert gas is supplied from the sterilizing-gas remover 17 which will be described later. Therefore, moisture or the like that remains after the integrity testing is removed, and it is possible to carry out removal of moisture inside the sterilization region S and drying thereof.

Furthermore, the valves 19Ab and 19Ac are in a closed state, the valves 19Aa, 19Ad, and 14c are in an opened state, and a purge gas such as inert gas is supplied from the sterilizing-gas remover 17. Accordingly, the moisture or the like that remains after the integrity testing is removed, it is possible to carry out removal of moisture inside the sterilization region S, particularly, inside the sterilization filter 14d and the supply pipe 14, and drying thereof.

Note that, as “integrity testing” of the invention, a different method depending on a filter to be utilized is adopted.

For the “integrity testing” of the invention, as described on 6.3 (1) (b) on third page of JISK3835, “based on JISK3832, JISK3833, or the instruction manual of the employed filter, an integrity testing is aseptically carried out and no defects such as damage to a test filter are checked.” is important.

That is, the invention is characterized in that an integrity testing device is additionally attached to a sterilized-liquefied gas apparatus that is not provided with an integrity testing device. Regarding this filter, the configuration of “the integrity testing is carried out in accordance with the method specified by JISK3832, JISK3833, or the instruction manual of the employed filter” is required for the invention.

That is, as particularly described, it is important to provide the configuration in which the filter portion is separated from the system of the apparatus (the sterilized-liquefied gas apparatus 10) and the sterilized-liquefied gas apparatus 10 carries out the integrity testing. For example, in a conventional configuration including the sterilization filter 14d, in FIG. 1, the valve 19Aa that closes an upstream pipe and the valve 19Ab that closes a downstream pipe are necessary, and a configuration including a blank flange that can disconnect and connect a flow passage directed from the sterilization filter 14d toward an integrity testing equipment 19B is necessary.

Additionally, as configuration to be provided, preferably, a configuration including the valves 19Ad and 19Ac (drain valve) functioning as a discharge port is adopted.

In the configuration of the sterilized-liquefied gas apparatus 10 shown in FIG. 1, the integrity testing equipment 19B is built in the sterilized-liquefied gas apparatus 10. However, since the integrity testing is used as “periodic inspection”, the integrity testing equipment 19B is not an equipment necessary for the sterilized-liquefied gas apparatus 10 at all times. Note that, since arrangement of a pipe and a valve provided at the pipe which are connected to a filter 18b is the same as arrangement of a pipe and a valve provided at the pipe which are connected to the aforementioned sterilization filter 14d, a description thereof is omitted.

However, the configuration in which the integrity testing equipment 19B is provided at the sterilized-liquefied gas apparatus 10 at all times is further safe. For this reason, in the following description, the case will be described in which the integrity testing equipment 19B is provided at the sterilized-liquefied gas apparatus 10 at all times.

Here, the annex described on sixth page of JISK3833 is referred. For example, the case of utilizing JISK3833 as the integrity testing be described below.

“2. Diffusive Flow Test” described in JISK3833 is basically similar to the description after paragraph 0045. Consequently, the detail is preferably reflected by the description of JISK3833.

By the steps of “7.1. (2)” described in JISK3833, the entirety of the filter is moisturized. Regarding the steps, in a state where deionized water is supplied from the integrity testing device side and the valve 19Ac is closed or slightly opened, the primary side is filled with the deionized water, and therefore “7.1. (2) (e)” is completed.

Next, the deionized water is discharged from the valve 19Ac and the valve 19Ad which are in a sealed state and “7.1. (2) (g)” is completed.

Thereafter, by performing “7.2. (1)”, the integrity testing is carried out.

After the integrity testing is completed, the device may be removed. Since the integrity testing device side is in a state of being connected to a flange, it is completed with the configuration in which a sealing side and drain side of two valves and top and bottom of two flow passage valves remain.

In paragraph 0046, although bacteria inspection is carried out while maintaining an aseptic condition, normally, the integrity testing may be only carried out. The test in accordance with JISK3835 or JISK3836 may be carried out as needed.

The sterilizer 16 can supply a sterilizing gas, for example, hydrogen peroxide, an ethylene oxide gas, or the like to the sterilization region S and is connected to the supply pipe 14 that is split through the valve 14b. As the sterilizer 16, a sterilizing gas supplier may be adopted which can generate a sterilizing gas such as a hydrogen peroxide gas or an ethylene oxide gas. As a specific configuration of the sterilizer 16, for example, a configuration utilizing a low-temperature gas plasma method using hydrogen peroxide which applies high-frequency or micro wave energy to a hydrogen peroxide gas under high vacuum and supplies 100%-electrolytic dissociation (ionization) gas, that is, a plasma gas, or a configuration utilizing low-temperature hydrogen peroxide gas sterilization (vaporized hydrogen peroxide sterilization method) which vaporizes hydrogen peroxide by a heating vaporization device and supplies the vapor is applicable.

Furthermore, as a sterilizing gas, the type of the sterilizing gas compliant with the standard of the supply object to which a sterilizing gas is supplied from the sterilizer 16 can be used, and not only hydrogen peroxide gas but also a gas such as ethylene oxide, propylene oxide, formaldehyde, ozone, NO₂ or the like can be used.

By opening the valve 14b, closing the valve 14a and the valve 14c, and supplying a sterilizing gas to the inside of the sterilized-liquefied gas apparatus 10 by the sterilizer 16, it is possible to gas-sterilize the sterilization region S located further downstream than the sterilization filter 14d.

Moreover, as the sterilizer 16, the inside of the sterilization region S is depressurized by a pumping device such as a vacuum pump, a substance (gas, liquid, or the like) inside the sterilization region S is discharged, and it is also possible to assist introduction of the sterilizing gas into the sterilization region S.

In this case, in order to spread the sterilizing gas over the entirety of the sterilization region S, a pumping device such as a vacuum pump or the like can be provided at the downstream side of the connection pipe 18, for example, at the downstream position of the valve 18a, or at the downstream position (outside position) of a valve 18c, or a pump can also be connected to these positions. As described above, a remaining matter inside the sterilization region S is replaced with the sterilizing gas by vacuuming the inside of the sterilization region S, a concentration of the sterilizing gas thereby increases, and therefore it is possible to uniformly sterilize the inside of the sterilization region S.

The sterilizing-gas remover 17 supplies a purge gas such as inert gas to the inside of the sterilized-liquefied gas apparatus 10, thereby removes the sterilizing gas, moisture generated by gas sterilization, or the like which remain after gas sterilization, removes a gas from the inside of the sterilization region S, and dies it. As the inert gas to be supplied, for example, nitrogen gas may be used. Note that, moisture inside the sterilization region S can be removed along with removal of the remaining sterilizing gas; however, in the case where further drying is necessary, it is preferable that nitrogen gas having the flow rate greater than the supply amount of the source gas of the liquefied gas can be supplied.

Moreover, as the sterilizing-gas remover 17, the inside of the sterilization region S is depressurized by a pumping device such as a discharge pump, a substance (gas, liquid, or the like) inside the sterilization region S is discharged, it is possible to additionally remove the sterilizing gas in the sterilization region S.

In this case, in order to remove the sterilizing gas from the entirety of the sterilization region S, a pumping device such as a pump or the like can be provided at the downstream side of the connection pipe 18, for example, at the downstream position of the valve 18a, or at the downstream position (outside position) of the valve 18c, or a pump can also be connected to these positions.

The upstream side of the connection pipe 18 (supplier) penetrates through the lid 11a serving as an upper end of the liquefied gas reservoir 11 and is located in an axial direction at the center of the liquefied gas reservoir 11, and the connection pipe 18 is provided so as to longitudinally extend into the inside of the liquefied gas reservoir 11.

It is only necessary that, when a liquefied gas is supplied to the liquefied gas reservoir 11, the upstream end portion of the connection pipe 18 is maintained at the position lower than a liquid level of the liquefied gas stored in the liquefied gas reservoir 11, and the liquefied gas stored in the liquefied gas reservoir 11 can be supplied to the outside through an output port via the connection pipe 18. Particularly, it is preferable that the upstream end portion of the connection pipe 18 be disposed to substantially come into contact with the reservoir recess 11d.

Furthermore, the upstream end portion of the connection pipe 18 may be disposed to substantially come into contact with the center portion that is the minimal position of the reservoir recess 11d. As the upstream end portion of the connection pipe 18 is disposed as mentioned above, when sterilization is carried out, the connection pipe 18 can also be used as a drain that can discharge moisture or the like stored in the reservoir recess 11d to the outside.

The valve 18a is provided at the position that is an output port supplying the stored liquefied gas from the liquefied gas reservoir 11 to the outside from the downstream side of the connection pipe 18, and the connection pipe 18 and the liquefied gas reservoir 11 can be hermetically-sealed with respect to the outside.

A flow passage (second flow passage) branched from a flow passage (first flow passage) that is from the liquefied gas reservoir 11 to the valve 18a is provided at the upstream side of the valve 18a of the connection pipe 18 and at the position between the valve 18a and the liquefied gas reservoir 11. A valve 19Ae, the filter 18b, and the valve 18c which will be described later are provided at the branched flow passage (second flow passage).

The downstream of the valve 18c is communicated with the outside, lets out a vaporizing superfluous gas to the outside when the liquefied gas is stored in the liquefied gas reservoir 11, and is provided so that an internal pressure of the liquefied gas reservoir 11 does not increase. Additionally, since the valve 18c is provided via the filter 18b, even in the case where an external air enters the connection pipe 18, contaminant such as harmful bacteria with respect to the connection pipe 18 can be prevented from entering the inside of the sterilization region S. Moreover, the valve 18c may be configured to be operated as a safety valve that operates in the case where an internal pressure of the liquefied gas reservoir 11 increases to be greater than or equal to a predetermined value.

The filter 18b may be provided to be connectable to the integrity testing device 19A that is connected to the aforementioned integrity testing unit 19B and serves as a device that checks and inspects that the filter is in a state of being capable of maintaining an aseptic condition.

The integrity testing device 19A with respect to the filter 18b may include: the valve 19Ae that is split from the connection pipe 18 and provided at the upstream side of the filter 18b; a valve 19Af that is provided so as to be split from a pipe between the valve 19Ae and the filter 18b; a valve 19Ag that is provided so as to be split from a pipe between the valve 18c and the filter 18b; a pipe 19Aw that is connected to the integrity testing unit 19B at the branching position of the valve 19Af; and a valve 19Av that connects the pipe 19Aw to the downstream position of the valve 19Ab of the supply pipe 14.

In the integrity testing device 19A with respect to the filter 18b, the valves 19Ae, 19Av, and 18c are in a closed state, the valves 19Af and 19Ag are in an opened state, the integrity testing unit 19B supplies, for example, deionized water, and deionized water that passed through the filter 18b is collected from the valve 19Ag. It is determined whether or not the filter maintains an aseptic condition by inspecting the collected deionized water.

After the integrity testing is completed, the valves 19Ae and 18c are in a closed state, the valve 19Af, 19Ag, 19Av are in an opened state, the valve 14c is opened, and a purge gas such as inert gas is supplied from the sterilizing-gas remover 17 which will be described later. Therefore, moisture or the like that remains after the integrity testing is removed, and it is possible to carry out removal of moisture of the connection pipe 18 or the like and drying thereof.

Furthermore, the integrity testing with respect to the sterilization filter 14d and the integrity testing with respect to the filter 18b can be carried out at the same time.

The output port of the connection pipe 18 which is the downstream side of the valve 18a is directly connected via a pipe to a region such as an isolator that uses a liquefied gas in an aseptic condition in the fields of medicine, pharmaceuticals, food, research, or the like, and it is possible to supply a sterilized-liquefied gas that is taken out from the output port to the outside.

At the liquefied gas reservoir 11, a pressure detecting device that measures an internal pressure, a temperature detecting device that measures an internal temperature, an internal-state display device 11f that displays detection results thereof, and the like are provided. As stated above, all of the devices that are necessary to be communicated with the outside of the liquefied gas reservoir 11 are disposed so as to penetrate through the lid 11a serving as an upper end of the liquefied gas reservoir 11.

Note that, regarding the temperature detecting device, as a plurality of positions which are not shown in the figure, can be provided inside the liquefied gas reservoir 11, and at which the temperature detecting device is disposed, for example, the upper position of the side wall of the liquefied gas reservoir 11, the position which is near the cooling unit 13a and at which a temperature of the cooling unit 13a can be detected, the position which is near the bottom portion 11b serving as a lower end of the liquefied gas reservoir 11, or the like is adopted.

In the sterilized-liquefied gas apparatus 10 according to the embodiment, the supply pipe 14 serving as the sterilization region S, the sterilization filter 14d, the liquefied gas reservoir 11, the cooling unit 13a, the connection pipe 18 communicated with the valve 18a, the connection pipe 18 communicated with the valve 18c, the pressure detecting device, and the top surface or the inner surface of the temperature detecting device are configured so as to satisfy sanitary specification.

For example, stainless steels, particularly, SUS316 or SUS316L is adopted as sanitary specification. Furthermore, by carrying out a specular finishing treatment that polishes the surface of stainless steels by use of an abrasive of No. 400 and by carrying out an electrochemical polishing treatment, it is possible to achieve the specification in compliance with a stainless steel sanitary pipe or the like which is determined by JIS standards. Alternatively, the configuration may be adopted in which the surface of the aforementioned stainless steels or the surface which was subjected to the aforementioned treatment is coated with Au, Pt, or the like.

Moreover, for Sanitary Standard, silicone, fluorine resin, fluorine rubber such as vinylidene fluoride (FKM), or the like can be used as the above-mentioned joints of pipes, O-rings, or the like.

Hereinafter, a method of producing a sterilized-liquefied gas in the sterilized-liquefied gas apparatus according to the embodiment will be described.

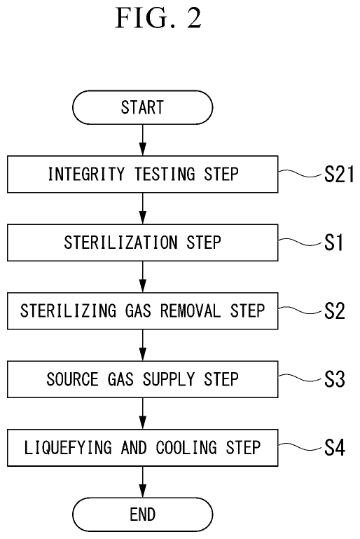

FIG. 2 is a flowchart showing steps of producing a liquefied gas in the sterilized-liquefied gas apparatus according to the embodiment.

As shown in FIG. 2, the method of producing a sterilized-liquefied gas in the sterilized-liquefied gas apparatus 10 according to the embodiment includes: an integrity testing step S21, a sterilization step S1, a sterilizing gas removal step S2, a source gas supply step S3, and a liquefying and cooling step S4. Note that, the integrity testing step S21 may also be omitted; however, in the case of carrying out the integrity testing step S21, the integrity testing step is carried out at a predetermined periodic timing and is not always an essential step.

In the method of producing a sterilized-liquefied gas in the sterilized-liquefied gas apparatus 10 according to the embodiment, first of all, as the sterilization step S1 shown in FIG. 2, the sterilization region S is sterilized.

In the sterilization step S1, firstly, the valve 14a and the valve 14c are in a closed state, the valve 14b is in an opened state, the cooler 13 is in a stopped state, the source-gas supplier 12 is in a stopped state, the valve 18a is in an opened state, and the valve 18c is in an opened state.

In this state, the sterilizer 16 is operated, a sterilizing gas serving as a hydrogen peroxide gas supplied from a sterilizing gas supplier and having approximately 100° C. is supplied to the supply pipe 14 that is split via the valve 14b. The sterilizing gas passes through the supply pipe 14 serving as the sterilization region S, the sterilization filter 14d, the liquefied gas reservoir 11, and the connection pipe 18 via the valve 14b, passes through the output port located at the downstream side of the connection pipe 18 communicated with the valve 18a and the filter 18b provided at the branched flow passage (second flow passage), and is discharged from the valve 18c. For this reason, the sterilizing gas comes into contact with the inner surfaces of the sterilization region S, the inner surfaces of the sterilization region S with which the sterilizing gas is in contact is subjected to sterilization treatment, and bacteria is annihilated from the inner surfaces of the sterilization region S.

As treatment conditions in the sterilization step S1, in order to completely obtain an aseptic condition, the sterilization conditions can be adopted which are required for, for example, a freezing dryer used for medicinal products. In such treatment conditions, the sterilization region S is exposed to the hydrogen peroxide gas for approximately 30 minutes to 45 minutes and the exposure state is maintained, and therefore bacteria is annihilated.

Furthermore, in the case of provision of a pumping device such as a vacuum pump as the sterilizer 16, the valve 18a is in a closed state, the valve 18c is in a closed state, the inside of the sterilization region S is depressurized, a substance inside the sterilization region S is discharged, thereafter the sterilizer 16 is operated, and it is also possible to assist introduction of the sterilizing gas into the sterilization region S.

The state where the sterilization region S exposed to the sterilizing gas is maintained for a predetermined amount of time is checked, operation of the sterilizer 16 is stopped, and the sterilization step S1 is completed.

Next, in the sterilizing gas removal step S2 shown in FIG. 2, the valve 14b is in a closed state, the valve 14c is in an opened state, the valve 18a is in an opened state, and the valve 18c is in an opened state.

In this state, the sterilizing-gas remover 17 is operated, inert gas having a temperature higher than a room temperature, preferably 100° C. or higher, is supplied to the supply pipe 14. For example, inert gas such as nitrogen gas passes through the supply pipe 14 serving as the sterilization region S, the sterilization filter 14d, the liquefied gas reservoir 11, and the connection pipe 18 via the valve 14c and is discharged from located near the output port the valve 18a of the lower end of the discharge pipe 18 and the lower end of a branch pipe (second flow passage) located near the valve 18c. Consequently, the sterilizing gas used for gas sterilization of the sterilization step S1 is discharged and removed, and the inside of the sterilization region S is purified and dried.

In the sterilizing gas removal step S2, since it is necessary to dry the inside of the sterilization region S such as the liquefied gas reservoir 11, the sterilizing-gas remover 17 supplies high-temperature inert gas having a flow rate greater than that of supply of a source gas from the source-gas supplier 12 in the liquefying and cooling step S4 which will be described later.

The high-temperature inert gas supplied from the sterilizing-gas remover 17 passes through the sterilization filter 14d in an aseptic condition and is therefore supplied to the inside of the sterilization region S in an aseptic condition where bacteria is annihilated.

In the sterilizing gas removal step S2, moisture or the like adhered to the inner surfaces of the sterilization region S is vaporized and discharged from the connection pipe 18 by the high-temperature inert gas supplied from the sterilizing-gas remover 17. It is checked whether the moisture adhered to the inside of the liquefied gas reservoir 11 and the inner surfaces of the sterilization region S is completely discharged to the outside, operation of the sterilizing-gas remover 17 is stopped, and the sterilizing gas removal step S2 is completed.

As the integrity testing step S21 shown in FIG. 2, integrity testing with respect to the sterilization filter 14d and the filter 18b which are mentioned above is carried out in accordance with the steps disclosed in the above-described JIS.

Additionally, it is preferable that the integrity testing step S21 be carried out previous to the sterilization step S1. The reason for this is that, there is a concern that portions of the sterilization filter 14d and the filter 18b are damp, and it prevents incorporation of contamination from the outside due to open and close of the valves 19Ad and 19Ac (drain valve).

Next, in the source gas supply step S3 shown in FIG. 2, the valve 14c is in a closed state, and the valve 14a is in an opened state. At the downstream side of the connection pipe 18, the valve 18a located near the output port is in a closed state, and the valve 18c is in a closed state. In this state, the source-gas supplier 12 is operated, and nitrogen gas serving as a source gas is supplied to the liquefied gas reservoir 11.

At this time, the source gas supplied from the source-gas supplier 12 flows in the supply pipe 14 through the sterilization filter 14d into the liquefied gas reservoir 11; however, the region into which the source gas flows is the sterilization region S that is located further downstream than the sterilization filter 14d. Since the sterilization treatment by the sterilizer 16 is completely finished, the portion serving as the sterilization region S located further downstream than the sterilization filter 14d can be maintained in an aseptic condition.

Next, in the liquefying and cooling step S4 shown in FIG. 2, the cooler 13 is operated, the compressor 13b circulates helium gas between the compressor 13b and the cooling unit 13a, removes heat from the cooling unit 13a, and the heat is discharged to the outside by the water cooling unit 13c. Consequently, as the cooling unit 13a is cooled down which penetrates through the lid 11a and protrudes toward the inside of the liquefied gas reservoir 11, the inside of the liquefied gas reservoir 11 is cooled down, and the source gas is liquefied.

The liquefied source gas is stored inside the liquefied gas reservoir 11.

By pressurizing the inside of the liquefied gas reservoir 11 by the sterilizing-gas remover 17 or the like as needed, the sterilized-liquefied gas stored in the liquefied gas reservoir 11 can be applied from the output port to the outside through the valve 18a in an opened state.

Here, while the valve 18a located near the output port or the valve 18c is in an opened state, it is also possible to supply a source gas serving as a raw material of a liquefied gas to the liquefied gas reservoir 11. The reason for this is that, the inside of the liquefied gas reservoir 11 has a pressure higher than atmospheric pressure in the sterilizing gas removal step S2 (1 atm+α) and has a state where a temperature thereof is also high. The reason for this is that the cooler 13 is not promptly cooled down during operation, and the internal pressure of the liquefied gas reservoir 11 increases until reaching the supply pressure of the source-gas supplier 12.

At the same time, in order to prevent bacteria from entering to the liquefied gas reservoir by setting the gas flow to be directed in a single direction, supply of the source gas by the source-gas supplier 12 is adjusted so that the inside pressure of the liquefied gas reservoir 11 is atmospheric pressure+α.

Alternatively, in the case where the liquefying and cooling step S4 is started in a state where the valve 18a is in a closed state and the valve 18c is in an opened state, since the filter 18b is provided, there is no possibility that bacteria enters through the valve 18c.

Additionally, during the liquefying operation while supplying the source gas, it is maintained that the valve 18a located near the output port is in a closed state and the valve 18c provided at the branched flow passage is in an opened state.

Furthermore, in the case where the liquefied gas in a liquid state is stored in a state where production of the liquefied gas is not carried out without supply of the source gas, a pressure inside the liquefied gas reservoir 11 increases due to evaporation of the source gas by heat input; consequently, an internal pressure of the liquefied gas reservoir 11 is controlled to be lowered to a predetermined value by cooperatively operating a pressure detecting device which is not shown in the figure and the valve 18c. Moreover, in the case of operating the valve 18c as a safety valve, the opened state is maintained.

In the production of the sterilized-liquefied gas in the sterilized-liquefied gas apparatus 10 according to the embodiment, the sterilization region S is in an aseptic condition by the sterilization step S1 and the sterilizing gas removal step S2. Since this aseptic condition is maintained and the treatment of liquefying the source gas can be carried out, it is possible to produce a sterilized-liquefied gas. Furthermore, as stated above, since the aseptic condition is maintained, it is possible to guarantee an aseptic condition of the produced sterilized-liquefied gas.

In the embodiment, the sterilization filter 14d is in an aseptic condition by the sterilization step S1 and the sterilizing gas removal step S2. In this state, by supplying the source gas from the source-gas supplier 12 to the liquefied gas reservoir 11 through the supply pipe 14, the source gas in an aseptic condition is supplied to the inside of the liquefied gas reservoir 11 through the sterilization filter 14d, and therefore liquefying treatment can be carried out.

In the embodiment, the upstream end of the connection pipe 18 is provided at the position near the reservoir recess 11d such that the upstream end is in contact with the reservoir recess. For this reason, it is possible to supply the liquefied gas stored in the liquefied gas reservoir 11 for as long as possible. In addition, since the reservoir recess 11d is provided at the bottom surface of the bottom portion 11b of the liquefied gas reservoir 11, the liquefied gas is collected in line with an inclined surface formed on the bottom surface of the bottom portion 11b, and the stored liquefied gas can be supplied to the outside.

Hereinafter, a sterilized-liquefied gas apparatus according to a second embodiment of the invention will be described with reference to drawings.

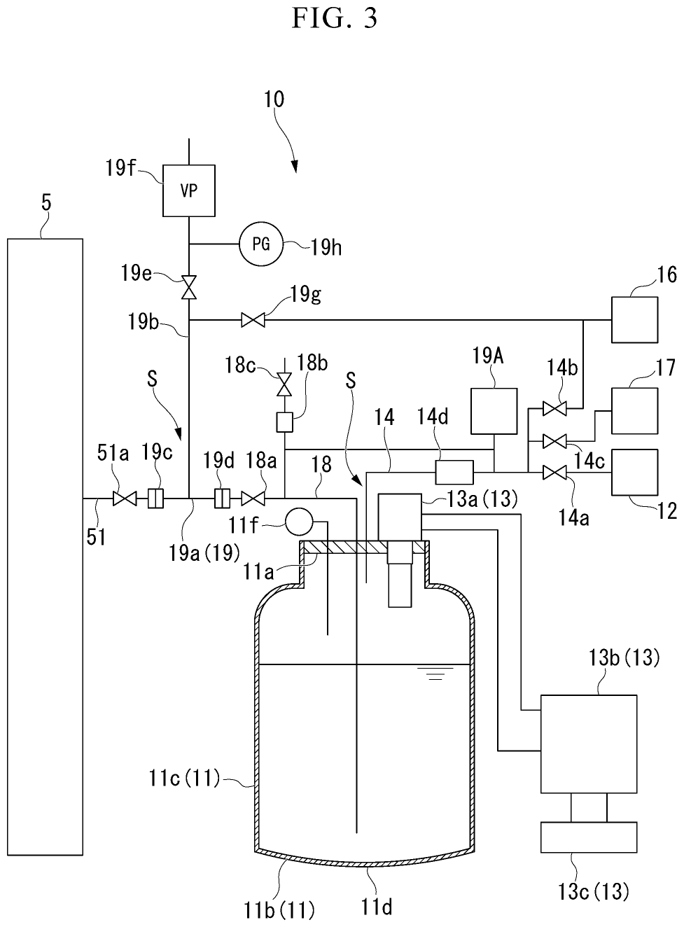

FIG. 3 is a schematic front view showing a sterilized-liquefied gas apparatus according to the embodiment. The embodiment is different from the aforementioned first embodiment in a supplier. In FIG. 3, identical reference numerals are used for the elements which are identical to those of the first embodiment, and the explanations thereof are omitted or simplified here.

In the embodiment, a connecting pipe 19 (supplier) is provided at the downstream side of the valve 18a of the connection pipe 18. The connecting pipe 19 connects the sterilized-liquefied gas apparatus 10 to an external device serving as a supply object to which a liquefied gas is supplied from the sterilized-liquefied gas apparatus 10. That is, the connecting pipe 19 has a function as a supplier. In the connecting pipe 19, in order to maintain a sterilized state of a device serving as the supply object (other device) and a liquefied gas to be supplied, sterilization treatment is carried out.

Note that, in FIG. 3, part of the integrity testing device 19A is omitted. As the configuration of the integrity testing device 19A shown in FIG. 3, the same configuration as the configuration shown in FIG. 1 is adopted.

As shown in FIG. 3, one end of the connecting pipe 19 is connected to the downstream side of the valve 18a of the connection pipe 18. The other end of the connecting pipe 19 includes: a main pipe 19a connected to a pipe 51 of an isolator 5 (liquefied-gas supply object) to which the liquefied gas stored in the liquefied gas reservoir 11 is supplied; and a branch pipe 19b branched from the main pipe 19a.

The main pipe 19a includes clamps 19c and 19d (connection member) located at both ends of the main pipe 19a. The clamp 19c is connectable to the front-end of the pipe 51 of the isolator 5. The clamp 19d is connectable to the lower end (output port) of the connection pipe 18 connected to the liquefied gas reservoir 11.

Both of the clamps 19c and 19d are each one-touch clamp and are simply removable. A joint formed of a sanitary ferrule is used inside the connection pipe between the main pipe 19a and the pipe 51 and inside the connection pipe between the main pipe 19a and the connection pipe 18 so that a step difference does not occur.

Note that, a connection sensor that detects a connecting condition when the connecting pipe 19 is connected to the pipe 51 is provided at the clamp 19c. As the sensor, specifically, a configuration may be used which uses: a proximity sensor; or applies a voltage of approximately 5V to a pipe and thereby detects an electrical current when the connection.

One end of the branch pipe 19b is communicated with the main pipe 19a, and a vacuum pump 19f is connected to the other end of the branch pipe 19b via a valve 19e. The vacuum pump 19f depressurizes an internal space of the connecting pipe 19 and thereby can discharge a gas inside the connecting pipe 19.

Moreover, a device that can supply a sterilizing gas via a valve 19g, particularly, a measurement device 19h that is connected to the sterilizer 16 and measures a degree of vacuum of the inside of the branch pipe 19b is provided at the branch pipe 19b. Regarding the measurement device 19h, as a measurement device which is not involved to a sterilizer, a diaphragm gauge is preferably used.

The aforementioned connecting pipe 19 is configured so that the surface thereof and the inner surfaces satisfy sanitary specification.

For example, stainless steels, particularly, SUS316 or SUS316L is adopted as sanitary specification. Furthermore, by carrying out a specular finishing treatment that polishes the surface of stainless steels by use of an abrasive of No. 400 and an electrochemical polishing treatment, it is possible to achieve the specification in compliance with a stainless steel sanitary pipe or the like which is determined by JIS standards. Alternatively, the configuration may be adopted in which the surface of the aforementioned stainless steels or the surface which was subjected to the aforementioned treatment is coated with Au, Pt, or the like.

Moreover, for Sanitary Standard, silicone, fluorine resin, fluorine rubber such as vinylidene fluoride (FKM), or the like can be used as the above-mentioned joints of pipes, O-rings, or the like.

The connecting pipe 19, the valve 18a of the connection pipe 18, the clamps 19c and 19d, a valve 51a, the valves 19e and 19g, the vacuum pump 19f, the measurement device 19h, and the sterilizer 16 constitute a supply sterilizer.

The connecting pipe 19 according to the embodiment is used when the liquefied gas stored in the liquefied gas reservoir 11 is supplied to the isolator 5 or the like. Accordingly, in the sterilized-liquefied gas apparatus 10, when liquefied gas production processing is being carried out, the connecting pipe 19 may be or may not be connected to the sterilized-liquefied gas apparatus 10.

When the sterilized-liquefied gas apparatus 10 is connected to the isolator 5, it is necessary to connect the connecting pipe 19 to the connection pipe 18 in advance.

In this case, the connecting pipe 19 is connected to the connection pipe 18 by the clamp 19d. Note that, in the case where the sterilized-liquefied gas apparatus 10 is configured to be provided with the connecting pipe 19 at all times, the clamp 19d may not be provided. Furthermore, in the case where the connecting pipe 19 is connected to the sterilized-liquefied gas apparatus 10 in advance, it is possible to preliminarily sterilize the inside of the connecting pipe 19 by the sterilization step S1 of the above-mentioned liquefied gas production.

When the sterilized-liquefied gas apparatus 10 is connected to the isolator 5 by use of the connecting pipe 19 according to the embodiment, the connecting pipe 19 is connected to the pipe 51 by the clamp 19c.

In this state, in order to maintain an aseptic condition of a liquefied gas to be supplied, treatment that sterilizes the connecting pipe 19, the connection pipe 18, and the pipe 51 is carried out.

Hereinbelow, a sterilization method of the connecting pipe (supplier) of the sterilized-liquefied gas apparatus according to the embodiment will be described.

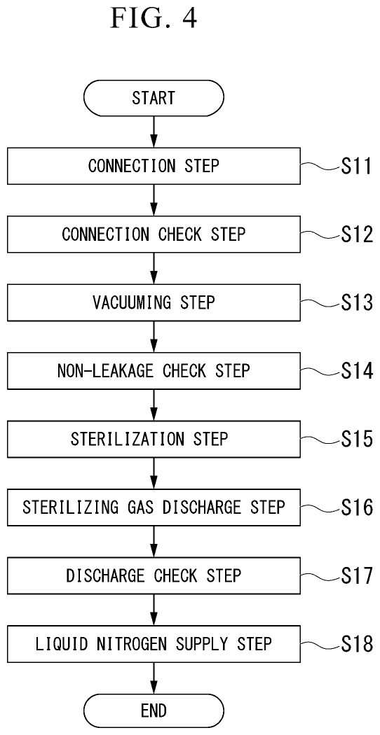

FIG. 4 is a flowchart showing a sterilization step in the supplier of the sterilized-liquefied gas apparatus according to the embodiment.

As shown in FIG. 4, the sterilization method of the sterilized-liquefied gas apparatus 10 according to the embodiment includes: a connection step S11; a connection check step S12; a vacuuming step S13; a non-leakage check step S14; a sterilization step S15; a sterilizing gas discharge step S16; a discharge check step S17; and a liquid nitrogen supply step S18.

In the sterilization method of the sterilized-liquefied gas apparatus 10 according to the embodiment, first of all, as the connection step S11 shown in FIG. 4, the connecting pipe 19 is connected to the pipe 51 of the isolator 5 by the clamp 19c of the sterilized-liquefied gas apparatus 10.

Note that, in the case where the connecting pipe 19 is not connected to the connection pipe 18, the connecting pipe 19 is connected to the connection pipe 18 by the clamp 19d.

In the connection step S11, firstly, the valve 18a, the valve 19g, the valve 19e, and the valve 51a are in a closed state, the vacuum pump 19f is in a stopped state, and the sterilizer 16 is in a stopped state. At this time, the source-gas supplier 12 is in a stopped state, the cooler 13 is in a stopped state, and the sterilizing-gas remover 17 is in a stopped state.

Next, as the connection check step S12 shown in FIG. 4, a connecting condition between the connecting pipe 19 and the pipe 51 is detected by a sensor provided at the clamp 19c of the connecting pipe 19. Subsequently, in a state where the signal cannot be detected, that is, in the case where connection between the connecting pipe 19 and the pipe 51 cannot be checked, it is configured not to proceed a subsequent step.

Next, as the vacuuming step S13 shown in FIG. 4, the inside of the connecting pipe 19 is depressurized, a gas inside the connecting pipe 19 is discharged. The reason for this is that, in the sterilization step S15 serving as a post-process, the entirety of the inside of the connecting pipe 19 is filled with the supplied sterilizing gas, a sufficient sterilization atmosphere is obtained, and moisture or the like that is present inside the connecting pipe 19 is discharged to the outside.

In the vacuuming step S13, the valve 19e is in an opened state, the vacuum pump 19f connected to the connecting pipe 19 is activated. The vacuum pump 19f is driven as a decompression discharge device.

As a result, the insides of the main pipe 19a and the branch pipe 19b separated by the valve 18a, the valve 51a, and the valve 19g are depressurized. The depressurized internal portion is the sterilization region S.

Next, as the non-leakage check step S14 shown in FIG. 4, while the valve 19e is in an opened state, a degree of vacuum of the inside of the branch pipe 19b is measured by the measurement device 19h, it is checked that a degree of vacuum of the inside of the connecting pipe 19 is in a state where it reaches a predetermined value. Accordingly, it is checked that leakage has not occurred inside the connecting pipe 19 and inside the sterilization region S, that is, connection of a joint, sealing due to a valve, or the like has been reliably carried out.

In the case where it is necessary to further reliably carry out the non-leakage check step S14, it is only necessary that a valve which is not shown in the figure is provided at the portion located directly above the air intake side of the vacuum pump 19f, the valve is closed after the vacuuming step S13 is carried out, and a space including the inside of the branch pipe 19b is a closed space. If it is not a closed space, an indicated value of the measurement device 19h shows that a pressure gradually increases with the passage of time due to a differential pressure between the internal pressure of the branch pipe 19b and an atmospheric pressure, and therefore the presence or absence of leakage can be determined. Since such method of determining the presence or absence of leakage is an integration method by use of a dynamic checking method of checking a reached pressure in a state where the vacuum pump 19f is continuously driven, it is possible to carry out leak determination with a high level of accuracy.

In the case where a state where a degree of vacuum of the inside of the connecting pipe 19 reaches a predetermined value cannot be checked, it is configured not to proceed a subsequent step.

If a state where a degree of vacuum of the inside of the connecting pipe 19 reaches a predetermined value can be checked, the valve 19e is in a closed state, the vacuum pump 19f is in a stopped state, and the non-leakage check step S14 is completed.

Next, as the sterilization step S15 shown in FIG. 4, the sterilization region S is sterilized.

In the sterilization step S15, the valve 18a is in a closed state, the valve 51a is in a closed state, the valve 19e is in a closed state, and the valve 19g is in an opened state.

In this state, the sterilizer 16 is operated, a sterilizing gas serving as a hydrogen peroxide gas supplied from a sterilizing gas supplier and having approximately 100° C. is supplied to the main pipe 19a of the connecting pipe 19 and the branch pipe 19b split from the main pipe 19a via the valve 19g.

Here, since the main pipe 19a and the inside of the branch pipe 19b which are separated by the valve 18a, the valve 51a, and the valve 19e are in a state being depressurized as the sterilization region S, the depressurized inside thereof is filled with the sterilizing gas. For this reason, the sterilizing gas comes into contact with the inner surfaces of the sterilization region S, the inner surfaces of the sterilization region S with which the sterilizing gas is in contact is subjected to sterilization treatment, and bacteria is annihilated from the inner surfaces of the sterilization region S.

As treatment conditions in the sterilization step S15, in order to completely obtain an aseptic condition, the sterilization conditions can be adopted which are required for, for example, a freezing dryer used for medicinal products. In such treatment conditions, the sterilization region S is exposed to the hydrogen peroxide gas for approximately 30 minutes to 45 minutes and the exposure state is maintained, and therefore bacteria is annihilated. Obviously, it is preferable that a valve which is not shown in the figure be provided at the portion located directly above the air intake side of the vacuum pump 19f, that a sterilizing gas be introduced into the sterilization region S in a vacuum state by closing the valve, and that a state where the sterilizing gas is stored be maintained.

The state where the sterilization region S exposed to the sterilizing gas is maintained for a predetermined amount of time is checked, operation of the sterilizer 16 is stopped, and the sterilization step S15 is completed.

Next, as the sterilizing gas discharge step S16 shown in FIG. 4, the valve 19g is in a closed state, and the valve 19e is in an opened state.

In this state, the vacuum pump 19f serving as a sterilizing gas discharger is activated, the sterilizing gas filling the inside of the connecting pipe 19 is discharged to the outside. In this situation, although not shown in the drawing, the concentration of a sterilizing gas remaining inside the connecting pipe 19 can be significantly lowered by filling (replacing) the sterilization region S (the inside of the connecting pipe 19) with air or inert gas instead of the sterilizer 16. Therefore, it is preferable that the sterilized-liquefied gas apparatus 10 be provided with a dilution gas introduction device and the sterilization step include a dilution step.

Next, as the discharge check step S17 shown in FIG. 4, the vacuum pump 19f serving as a sterilizing gas discharger is stopped, while the valve 19e is in an opened state, a degree of vacuum of the inside of the branch pipe 19b is measured by the measurement device 19h. Based on the measurement result, it is checked that a degree of vacuum of the inside of the connecting pipe 19 is in a state where it reaches a predetermined value, and accordingly it is checked that the sterilizing gas is discharged from the inside the connecting pipe 19 and the sterilization region S.

At the same time, it is checked that leakage has not occurred inside the sterilization region S, that is, connection of a joint, sealing due to a valve, or the like has been reliably carried out.

Here, in the case where a state where a degree of vacuum of the inside of the connecting pipe 19 is maintained to a predetermined value cannot be checked, it is configured not to proceed a subsequent step.

Next, the valve 19g is in a closed state, the valve 19e is in a closed state, and sterilization treatment with respect to the connecting pipe 19 is completed.

After completion of the sterilization treatment with respect to the connecting pipe 19 can be checked, as the liquid nitrogen supply step S18 shown in FIG. 4, the valve 18a is in an opened state, and the valve 51a is in an opened state. In this state, aseptic liquid nitrogen stored in the liquefied gas reservoir 11 is supplied to the pipe 51 of the isolator 5 while maintaining and guaranteeing an aseptic condition.

In the sterilized-liquefied gas apparatus 10 according to the embodiment, in a way similar to the case of the above-described first embodiment, a sterilized-liquefied gas can be produced, and it is possible to carry out the sterilization treatment with respect to the connecting pipe 19 in a state where the produced aseptic liquid nitrogen (liquefied gas) is stored in the liquefied gas reservoir 11. Consequently, bacteria in the sterilization region S that is separated by the valve 18a, the valve Ma, the valve 19e, and the valve 19g is annihilated, in this state, it is possible to start the liquid nitrogen supply step S18.

Because of this, at an optional place at which the sterilized-liquefied gas apparatus 10 is used, before the sterilized-liquefied gas (liquid nitrogen) is supplied to an optional connection object to be connected to the sterilized-liquefied gas apparatus 10, aseptic treatment with respect to the connecting pipe 19 and the connection portion is carried out without using other facilities, and it is possible to easily supply the sterilized-liquefied gas to the liquefied-gas supply object (connection object).

Additionally, in the sterilized-liquefied gas apparatus 10 according to the embodiment, since the sterilization treatment with respect to the connecting pipe 19 includes the steps S12, S14, and S17 which checks each of the steps S11, S13, S15, and S16, the sterilization treatment with respect to the connecting pipe 19 can be automated. Therefore, only by connecting the connecting pipe 19 to the pipe 51 by an operator, it is possible to supply a liquefied gas to the liquefied-gas supply object in a state where sterilization treatment is completed, and liquefied gas supply can be achieved while maintaining and guaranteeing an aseptic condition.

Furthermore, since it is not necessary to provide a device for carrying out sterilization treatment at the isolator 5 serving as the liquefied-gas supply object, the sterilized-liquefied gas is easily supplied to the supply object such as a plurality of isolators 5 when necessary and as much as required.

Note that, in the embodiment, as a device that supplies the sterilizing gas to the connecting pipe 19, the sterilizer 16 that carries out gas sterilization for liquefied gas production is used. That is, the sterilizer 16 includes both a function of supplying the sterilizing gas to the sterilization region S of the first embodiment and a function of supplying the sterilizing gas to the connecting pipe 19. In the embodiment, a configuration may be adopted in which a sterilizer that is provided differently from the sterilizer 16 supplies a sterilizing gas to the connecting pipe 19.

Hereinafter, a sterilized-liquefied gas apparatus according to a third embodiment of the invention will be described with reference to drawings.

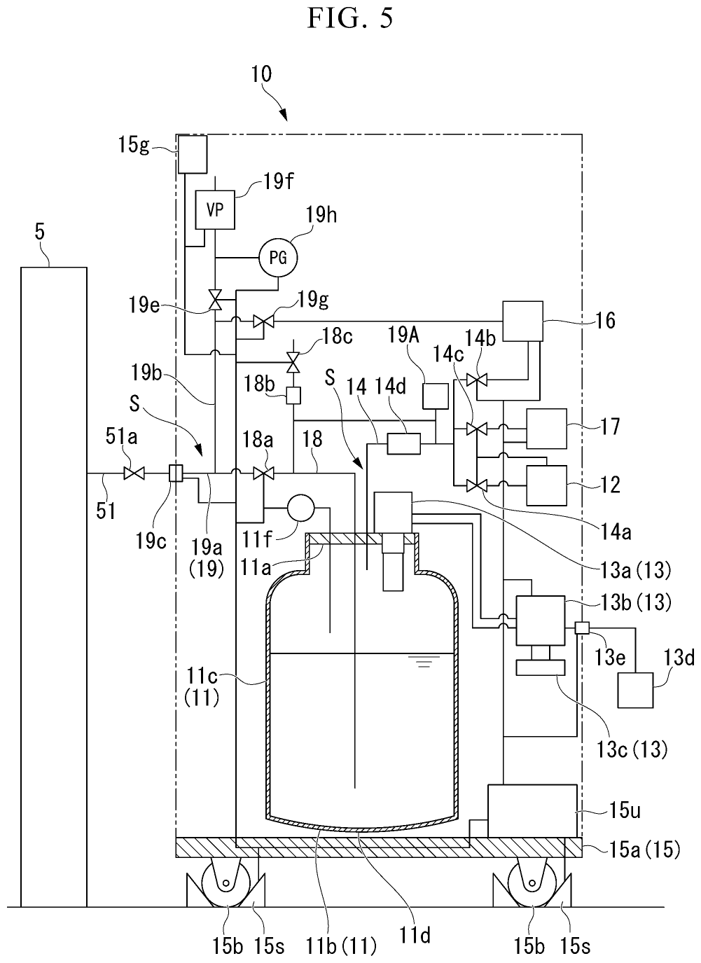

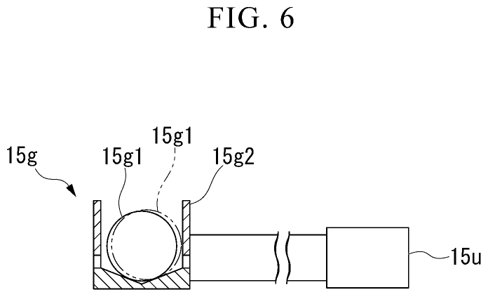

FIG. 5 is a schematic diagram showing a sterilized-liquefied gas apparatus according to the embodiment, and FIG. 6 is a schematic diagram showing an example of a moving sensor of the sterilized-liquefied gas apparatus according to the embodiment.

The embodiment is different in a transportable device from the above-described first and second embodiments. In FIGS. 5 and 6, identical reference numerals are used for the elements which are identical to those of the first and second embodiments, and the explanations thereof are omitted or simplified here.

As shown in FIG. 5, the sterilized-liquefied gas apparatus 10 according to the embodiment includes at least a carriage 15a, wheels 15b, a moving sensor 15g such as an acceleration sensor, and a controller 15u, which serve as a moving device 15 (transportable device) that is capable of moving the liquefied gas reservoir 11.

As shown in FIG. 5, the liquefied gas reservoir 11, the source-gas supplier 12, the cooler 13, the supply pipe 14, the controller 15u, the sterilizer 16, the sterilizing-gas remover 17, the liquefied gas supplier 18 (connection pipe), and the connecting pipe 19 are mounted on the carriage 15a. In FIG. 5, the carriage 15a can integrally move members (the aforementioned apparatus, valves, pipes, or the like) surrounded by a broken line. Note that, a support member that fixes the members surrounded by a broken line to the carriage 15a is not shown in the figure.

A plurality of wheels 15b are provided at the lower portion of the carriage 15a, and therefore the carriage 15a is movable. Each of the wheels 15b can be fixed to and removed from stoppers 15s provided on a floor or the like.

The stoppers 15s can fix the carriage 15a so that the sterilized-liquefied gas apparatus 10 is disposed at the position at which liquefied gas production is carried out and at the position at which the isolator 5 to which the produced liquefied gas is supplied is connectable to the connecting pipe 19. Accordingly, the carriage 15a is movable between the position at which liquefied gas production is carried out and the position at which liquefied gas supply is carried out and can carry out both of liquefied gas production and liquefied gas supply.

As shown in FIG. 5, a sensor that is connected to the controller 15u is provided at each stopper 15s. Only when the wheel 15b is located at the position corresponding to the stopper 15s, operation of liquefied gas production or operation of liquefied gas supply is carried out in the sterilized-liquefied gas apparatus 10 in accordance with a detection signal output from the sensor.

Particularly, as the sensor, a proximity sensor, a weight detection sensor, a contact detection sensor, or the like may be adopted.

The moving sensor 15g is integrally provided at the carriage 15a and can detect a state where the carriage 15a is being moved.

As the moving sensor 15g, a configuration of the moving sensor 15g is not limited as long as movement of the carriage 15a can be detected. For example, as shown in FIG. 6, a configuration may be adopted in which a ball 15g1 formed of a conductor is disposed in a case 15g2 having opening at the upper portion thereof. In this case, when the carriage 15a is moved, an electrically detected signal due to contact of the ball 15g1 to the inner wall of the case 15g2 as indicated by a broken line can be output to the controller 15u.

The moving sensor 15g is connected to the controller 15u, only when the moving sensor 15g does not detect the movement, operation of liquefied gas production or operation of liquefied gas supply can be carried out in the sterilized-liquefied gas apparatus 10.

The controller 15u is connected to: a pressure detecting device and a temperature detecting device which are connected to the internal-state display device 11f of the liquefied gas reservoir 11; the source-gas supplier 12; the cooler 13; the supply pipe 14; the valves 14a, 14b, and 14c; the sensor of the stopper 15s, the moving sensor 15g; the sterilizer 16; the sterilizing-gas remover 17; the valves 18a and 18c of the liquefied gas supplier 18 (connection pipe); the valves 19e and 19g of the connecting pipe 19, the vacuum pump 19f and the measurement device 19h; and a sensor of the clamps 19c and 19d. The controller 15u controls operations of the aforementioned devices or members or receives a signal output from the aforementioned members.

At the same time, the controller 15u is integrated with a power supply serving as a uninterruptible power source that carries out power supply necessary for the sterilized-liquefied gas apparatus 10 and the constituent members of the isolator 5 when the carriage 15a is in movement or liquefied gas is supplied to the isolator 5.

Furthermore, the controller 15u is also connected to a power supply 13d and a connector 13e which supply a power to the cooler 13 at the position at which liquefied gas production is carried out. The controller 15u controls operations of the cooler 13, the power supply 13d, and the connector 13e or receives a signal output from the aforementioned members.

The power required for operation of the cooler 13 is significantly greater than the power required for operation of the other configurations described above. For this reason, in the case of operating the cooler 13, power supply is carried out by the power supply 13d that is fixed to facilities and can supply a large amount of power but not by the power supply serving as the uninterruptible power source integrated with the controller 15u.

In the sterilized-liquefied gas apparatus 10 according to the embodiment, since operation of liquefying treatment is not carried out when the carriage 15a is in movement by each of the aforementioned sensors, it is possible to ensure safety.

Moreover, in the sterilized-liquefied gas apparatus 10 according to the embodiment, the configuration necessary for the sterilized-liquefied gas apparatus 10 is mounted on the carriage 15a. That is, the sterilized-liquefied gas apparatus 10 is portable, and the sterilized-liquefied gas apparatus 10 is movable to an optional place. For example, the sterilized-liquefied gas apparatus 10 can be moved to a plurality of the isolators 5. In this case, without using other facilities, before a liquefied gas is supplied to the isolator 5, aseptic treatment is carried out with respect to the connecting pipe 19 and the connection portion through which the liquefied gas flows, and supply of the sterilized-liquefied gas can be easily carried out. Furthermore, space-saving and weight saving of the portable sterilized-liquefied gas apparatus 10 can be achieved.

Additionally, since reduction in size and space-saving of the sterilized-liquefied gas apparatus 10 are achieved, the sterilized-liquefied gas apparatus 10 can be easily used in a small-scale facility such as a research facility in which a plurality of the isolators 5 are provided in the same room. In this case, a large-scale sterilization liquefied gas apparatus is not provided. Furthermore, it is not necessary to connect a sterilization liquefied gas production apparatus to a supply object, for example, a production apparatus that cannot be moved, via a long pipe. Regarding layout change of a facility such as movement of the isolator 5 or the like, without significant reconstruction, supply of the sterilized-liquefied gas can be easily carried out.

In addition, the carriage 15a causes the sterilized-liquefied gas apparatus 10 to be movable to the supply object to which a sterilized-liquefied gas is to be supplied, it is checked that required aseptic treatment is completed, while guaranteeing an aseptic condition of an optional supply object, supply of the sterilized-liquefied gas can be easily carried out.

In addition, in the sterilized-liquefied gas apparatus 10 according to the embodiment, a sterilized-liquefied gas can be produced in a way similar to the case of the above-described first embodiment, and it is possible to carry out the sterilization treatment with respect to the connecting pipe 19 in a state where the aseptic liquid nitrogen (liquefied gas) that is produced in a way similar to the case of the aforementioned second embodiment is stored in the liquefied gas reservoir 11. Accordingly, bacteria of the sterilization region S is annihilated, in this state, it is possible to start the liquid nitrogen supply step S18.

Moreover, in the sterilized-liquefied gas apparatus 10 according to the embodiment, since the connection processing and sterilization treatment with respect to the connecting pipe 19 include the steps S12, S14, and S17 which checks each of the steps S11, S13, S15, and S16 in a way similar to the case of the aforementioned second embodiment, the sterilization treatment with respect to the connecting pipe 19 can be automated. Therefore, only by connecting the connecting pipe 19 to the pipe 51 by an operator, it is possible to supply a liquefied gas to the liquefied-gas supply object in a state where sterilization treatment is completed, and liquefied gas supply can be achieved while maintaining and guaranteeing an aseptic condition.

Furthermore, since it is not necessary to provide a device for carrying out sterilization treatment at the isolator 5 serving as the liquefied-gas supply object, the sterilized-liquefied gas is easily supplied to the supply object such as a plurality of isolators 5 when necessary and as much as required while maintaining safety.

Note that, in the embodiment, as a device that supplies the sterilizing gas to the connecting pipe 19, the sterilizer 16 that carries out gas sterilization for liquefied gas production is used. That is, the sterilizer 16 includes both a function of supplying the sterilizing gas to the sterilization region S of the first embodiment and a function of supplying the sterilizing gas to the connecting pipe 19. In the embodiment, a configuration may be adopted in which a sterilizer that is provided differently from the sterilizer 16 supplies a sterilizing gas to the connecting pipe 19.

Moreover, in the embodiment, electric power is supplied from the fixed power supply 13d to the cooler 13. It is not limited to this configuration, the power supply 13d may be mounted on the carriage 15a. That is, a portable power supply that can carry out a large amount of power supply can be mounted on the carriage 15a.

Hereinafter, a sterilized-liquefied gas apparatus according to a fourth embodiment of the invention will be described with reference to drawings.

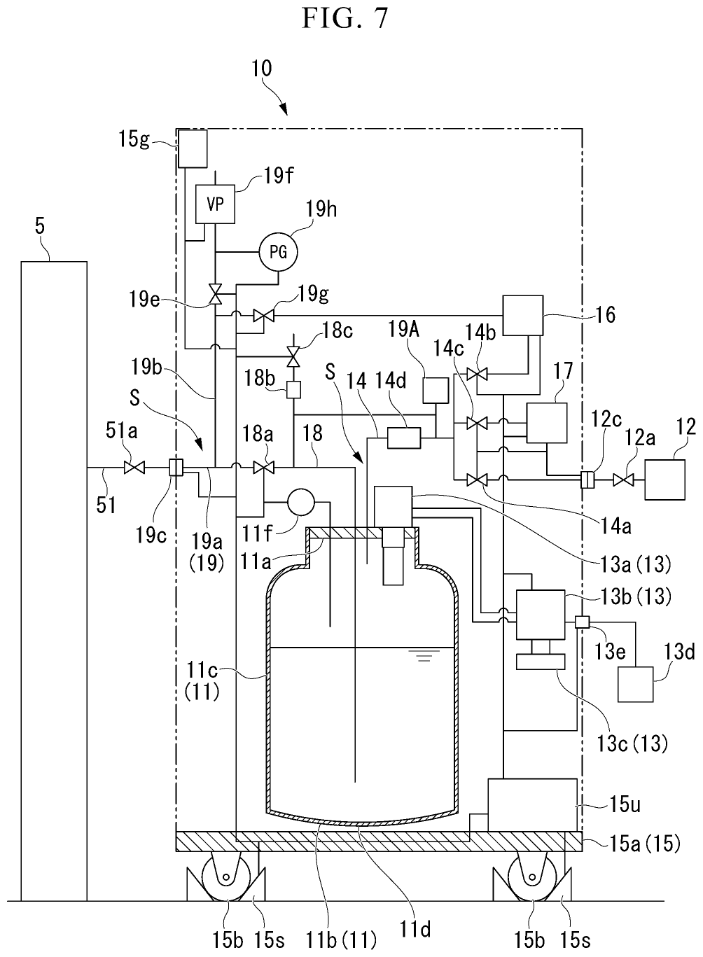

FIG. 7 is a schematic diagram showing a sterilized-liquefied gas apparatus according to the embodiment.

The embodiment is different in a configuration of a transportable device from the above-described first to third embodiments. In FIG. 7, identical reference numerals are used for the elements which are identical to those of the first to third embodiments, and the explanations thereof are omitted or simplified here.

As shown in FIG. 7, in the sterilized-liquefied gas apparatus 10 according to the embodiment, the source-gas supplier 12 is disposed in a state of being fixed in similar to the power supply 13d. Furthermore, regarding the supply pipe 14, a valve 12a and a clamp 12b are provided between the source-gas supplier 12 and the valve 14a.

As a configuration of the clamp 12b, the same configuration as those of the clamps 19c and 19d is adopted. The supply pipe 14 can be connected to and separated from the source-gas supplier 12. Similarly, as a configuration of the valve 12a, the same configuration as that of the valve 18a is adopted. The supply pipe 14 can be connected to and separated from the source-gas supplier 12. Additionally, a connection sensor is provided at the clamp 12b, detects that it is in a connected state, and can output the signal thereof to the controller 15u.

Note that, in the case where the source-gas supplier 12 is separated from and connected to the liquefied gas reservoir 11, in a manner similar to the aforementioned second and third embodiments, the configuration can that carry out sterilization in a way similar to the case of the connecting pipe 19 can also be disposed at the position near the clamp 12b.

In the embodiment, in the case of carrying out liquefied gas production, the liquefied gas production is carried out at the position (liquefied gas production position) near the source-gas supplier 12 that is disposed in a state of being fixed. In the case where the produced liquefied gas is supplied to the isolator 5 or the like, the sterilized-liquefied gas apparatus 10 moves to the position (liquefied gas supply position) near the isolator 5, the sterilized-liquefied gas apparatus 10 can also be connected to the isolator 5. Furthermore, since a configuration is adopted in which the power supply 13d and the source-gas supplier 12 are disposed in a state of being fixed and the power supply 13d and the source-gas supplier 12 are not mounted on the carriage 15a, space-saving and weight saving of the portable sterilized-liquefied gas apparatus 10 can be further achieved.

Note that, in each embodiment mentioned above, in order to realize space-saving and weight saving of the portable sterilized-liquefied gas apparatus 10, a device or members which are not mounted on the carriage 15a can be optionally selected.

Furthermore, the integrity testing step S21 of the first embodiment, after the sterilization treatment is carried out, regardless of whether or not the liquefied gas production processing is carried out, inspection of the filters 14d and 18b is periodically carried out at a predetermined intervals, and therefore it is preferable to maintain an aseptic condition. In the second to fourth embodiments, although there is no explanation regarding the integrity testing step S21, it is preferable to appropriately carry out the step.

While preferred embodiments of the invention have been described and shown above, it should be understood that these are exemplary of the invention and are not to be considered as limiting. Additions, omissions, substitutions, and other modifications can be made without departing from the scope of the present invention. Accordingly, the invention is not to be considered as being limited by the foregoing description, and is only limited by the scope of the appended claims.

### INDUSTRIAL APPLICABILITY

As application examples of the invention, provision of aseptic liquid nitrogen can be adopted which can be directly used for aseptic medicines or the like in the field of bio medicine and regenerative medicine or in the case where it is necessary to rapidly cool down a product in a freezing dryer or which can be directly used in an aseptic room.

### DESCRIPTION OF REFERENCE NUMERALS

10 sterilized-liquefied gas apparatus
11 liquefied gas reservoir
11d reservoir recess
11f internal-state display device
12 source-gas supplier
13 cooler (mechanical freezing machine system)
13a cooling unit
13b compressor
13c water cooling unit
13d power supply
13e connector
14 supply pipe
14a, 14b, 14bc valve
14d sterilization filter
15 moving device (transportable device)
15a carriage
15b wheel
15s stopper
15g moving sensor
15u controller (power supply)
16 sterilizer
17 sterilizing-gas remover
18 connection pipe (supplier)
18a, 18c valve
18b filter
19 connecting pipe (supplier)
19a main pipe
19b branch pipe
19c, 19d clamp
19e, 19g valve
19f vacuum pump
19h measurement device
19A integrity testing device
19Aa to 19Ag, 19Av valve
19B integrity testing unit
5 isolator
51 pipe
51a valve
S sterilization region

What is claimed is:

1. A sterilized-liquefied gas apparatus, comprising:
a liquefied gas reservoir;
a source-gas supplier that supplies a source gas to the liquefied gas reservoir;
a cooler that cools down an inside of the liquefied gas reservoir to liquefy the source gas;
a supply pipe that connects the source-gas supplier and the liquefied gas reservoir;
a sterilization filter provided at the supply pipe;
a sterilizer that sterilizes a sterilization region by a sterilizing gas, the sterilization region being located further downstream than the sterilization filter; and
a sterilizing-gas remover that removes the sterilizing gas after sterilization.

2. The sterilized-liquefied gas apparatus according to claim 1, wherein a liquefied gas that is obtained by liquefying the source gas is liquid nitrogen.

3. The sterilized-liquefied gas apparatus according to claim 1, further comprising:
a moving device that is capable of moving at least the liquefied gas reservoir.

4. The sterilized-liquefied gas apparatus according to claim 1, further comprising:
a supplier that is connected to the liquefied gas reservoir, supplies a liquefied gas stored in the liquefied gas reservoir toward a downstream side of the liquefied gas reservoir, and is capable of being hermetically-sealed; and
a supply sterilizer that sterilizes the supplier.

5. The sterilized-liquefied gas apparatus according to claim 4, wherein
the supply sterilizer is capable of supplying the liquefied gas to the liquefied-gas supply object in an aseptic condition when the supplier is connected to a liquefied-gas supply object, and
the supply sterilizer is capable of carrying out aseptic treatment that sterilizes a supplier.

6. The sterilized-liquefied gas apparatus according to claim 5, further comprising:
a connection sensor that detects that the supplier is connected to the liquefied-gas supply object, wherein
in the case where the connection sensor determines that the supplier is connected to the liquefied-gas supply object, the supply sterilizer is capable of starting the aseptic treatment.

7. The sterilized-liquefied gas apparatus according to claim 1, further comprising:
a moving sensor that detects that the liquefied gas reservoir is in movement, wherein
in the case where the moving sensor detects that the sterilized-liquefied gas apparatus is in movement, liquefying treatment is able to be stopped.

8. A connecting pipe to be connected to a liquefied gas reservoir and a liquefied-gas supply object, the connecting pipe comprising:
a connection portion that is connectable to the liquefied gas reservoir and the liquefied-gas supply object; and
a valve capable of being hermetically-sealed, wherein
in a state where an inside of the connecting pipe is hermetically sealed by the valve, the connecting pipe is connected to a vacuum pumping device that is capable of discharging gas inside the connecting pipe, and
the connecting pipe is connected to a sterilizer that is capable of supply a sterilizing gas to an inside of the connecting pipe in a state where gas is discharged therefrom.
